(19) United States
(12) Patent Application Publication
(10) Pub. No.: US 2020/0324783 A1
(43) Pub. Date: Oct. 15, 2020

(54) PERSONALIZED SEATING ZONE SYSTEM

(71) Applicant: KARMA AUTOMOTIVE LLC, Irvine, CA (US)

(72) Inventors: Michael GROENE, Laguna Niguel, CA (US); David WITT, Newport Beach, CA (US); Gareth WILLIAMS, Orange, CA (US); Andre Franco LUIS, Orange, CA (US); Andreas THURNER, Irvine, CA (US)

(73) Assignee: KARMA AUTOMOTIVE LLC, Irvine, CA (US)

(21) Appl. No.: 16/456,741

(22) Filed: Jun. 28, 2019

Related U.S. Application Data
(60) Provisional application No. 62/832,477, filed on Apr. 11, 2019.

(51) Int. Cl. B60W 50/00; B60N 2/02; G01C 21/34; B60H 1/00; A61B 5/1171; A61B 5/00; G06K 9/00

(52) U.S. Cl. CPC B60W 50/0098; B60N 2/0248; G01C 21/3484; G06K 9/00288; A61B 5/1176; A61B 5/6893; G06K 9/00617; B60H 1/00735

(57) ABSTRACT

A personalized seating zone system of a vehicle cabin and a method for implementing such. The personalized seating zone system includes at least one personalized seating zone and is configured to collect biometric data corresponding to an occupant of the personalized seating zone system, match the collected biometric data to an identification tag to identify the occupant by comparing the collected biometric data to stored biometric data from an identification database, and load seat system settings for a personalized seating zone within the personalized seating zone system based on the matched identification tag.

### CROSS-REFERENCE TO RELATED APPLICATIONS

This application claims priority to and the benefit of U.S. Provisional Patent Application No. 62/832,477 filed on Apr. 11, 2019. The foregoing provisional application is incorporated by reference herein in its entirety.

### BACKGROUND

The present disclosure relates to a personalized seating zone system of a vehicle cabin and a method for implementing such.

Modern vehicles include a large number of systems, with each system including a large amount of system settings for various options. Customizing these system settings allows for a more personalized experience while traveling, but different vehicle occupants often desire different settings. A number of options for storing and loading system settings exist, but these require vehicle occupants to spend significant time with a user interface before configuring the systems.

It is desirable to have a system for loading vehicle system settings that requires minimal input from vehicle occupants.

### SUMMARY

Disclosed herein is a personalized seating zone system of a vehicle cabin and a method for implementing such. In one embodiment, the personalized seating zone system includes at least one personalized seating zone. In one embodiment, the personalized seating zone system is configured to collect, by at least one biometric sensor, biometric data corresponding to an occupant of the personalized seating zone system. In one embodiment, the personalized seating zone system is configured to match the collected biometric data to an identification tag to identify the occupant by comparing the collected biometric data to stored biometric data from an identification database. In one embodiment, the personalized seating zone system is configured to load seat system settings for a personalized seating zone within the personalized seating zone system based on the matched identification tag.

In another disclosed embodiment, collecting biometric data includes collecting facial recognition data. In another disclosed embodiment, collecting biometric data includes collecting iris recognition data. In another disclosed embodiment, the identification database includes an iris recognition database and matching the collected biometric data includes comparing the collected iris recognition data to stored iris recognition data from an iris recognition database.

In another disclosed embodiment, the personalized seating zone system further bases the loading of seat system settings on a seating position of the occupant.

In another disclosed embodiment, the seat system settings include at least one from the set of phone system settings, navigation system settings, audio system settings, video system settings, climate control settings, browser settings, infotainment settings, and seating adjustment settings.

In another disclosed embodiment, the personalized seating zone system loads personal information based on the matched identification tag. In another disclosed embodiment, the personal information includes at least one from the set of settings history, browser history, and browser favorites.

In another disclosed embodiment, the personalized seating zone system loads cloud-based applications based on the matched identification tag. In another disclosed embodiment, the personalized seating zone system additionally loads settings for cloud-based applications.

Other aspects, features, and techniques will be apparent to one skilled in the relevant art in view of the following detailed description of the embodiments.

### BRIEF DESCRIPTION OF THE DRAWINGS

The features, objects, and advantages of the disclosed embodiments will become more apparent from the detailed description set forth below when taken in conjunction with the drawings in which like reference characters identify correspondingly throughout and wherein:

FIG. 1 is a perspective view of an exemplary embodiment of a vehicle in which a personalized seating zone system may be installed.

FIG. 2 is a cutaway perspective view of an exemplary embodiment of a vehicle with a personalized seating zone system including a plurality of personalized seating zones.

FIG. 3 is a graphical representation of an exemplary embodiment of a personalized seating zone.

FIG. 4 is a perspective view of an exemplary embodiment of a personalized seating zone.

FIG. 5 is a flow chart depicting an exemplary saving process for saving a user's biometric data and seat system settings to a plurality of databases for a personalized seating zone.

FIG. 6 is a flow chart depicting an exemplary loading process for loading a user's seat system settings from a plurality of databases for a personalized seating zone.

### DETAILED DESCRIPTION

One aspect of the disclosure is directed to a personalized seating zone system of a vehicle cabin.

References throughout this document to “one embodiment,” “certain embodiments,” “an embodiment,” or similar term mean that a particular feature, structure, or characteristic described in connection with the embodiment is included in at least one embodiment. Thus, the appearances of such phrases in various places throughout this specification are not necessarily all referring to the same embodiment. Furthermore, the particular features, structures, or characteristics may be combined in any suitable manner on one or more embodiments without limitation. For example, two or more of the innovative devices described herein may be combined in a single device, but the application is not limited to the specific exemplary combinations of personalized seating zones that are described herein.

As used herein, the terms “a” or “an” shall mean one or more than one. The term “plurality” shall mean two or more than two. The term “another” is defined as a second or more. The terms “including” and/or “having” are open ended (e.g., comprising). The term “or” as used herein is to be interpreted as inclusive or meaning any one or any combination. Therefore, “A, B or C” means “any of the following: A; B; C; A and B; A and C; B and C; A, B and C”. An exception to this definition will occur only when a combination of elements, functions, steps or acts are in some way inherently mutually exclusive.

A detailed description of various embodiments is provided; however, it is to be understood that the disclosed embodiments are merely exemplary and may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the disclosed embodiments.

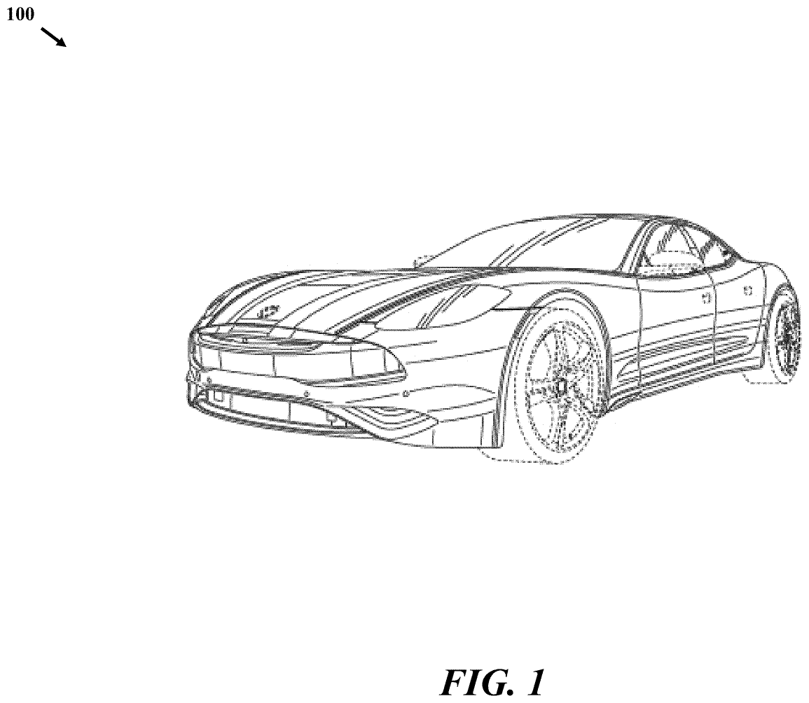

FIG. 1 is a perspective view of an exemplary embodiment of a vehicle 100 in which a personalized seating zone system 200 may be installed. The vehicle 100 shown in FIG. 1 is exemplary. The personalized seating zone system 200 described herein may be used with any passenger vehicle or other vehicle featuring customizable settings for its occupants.

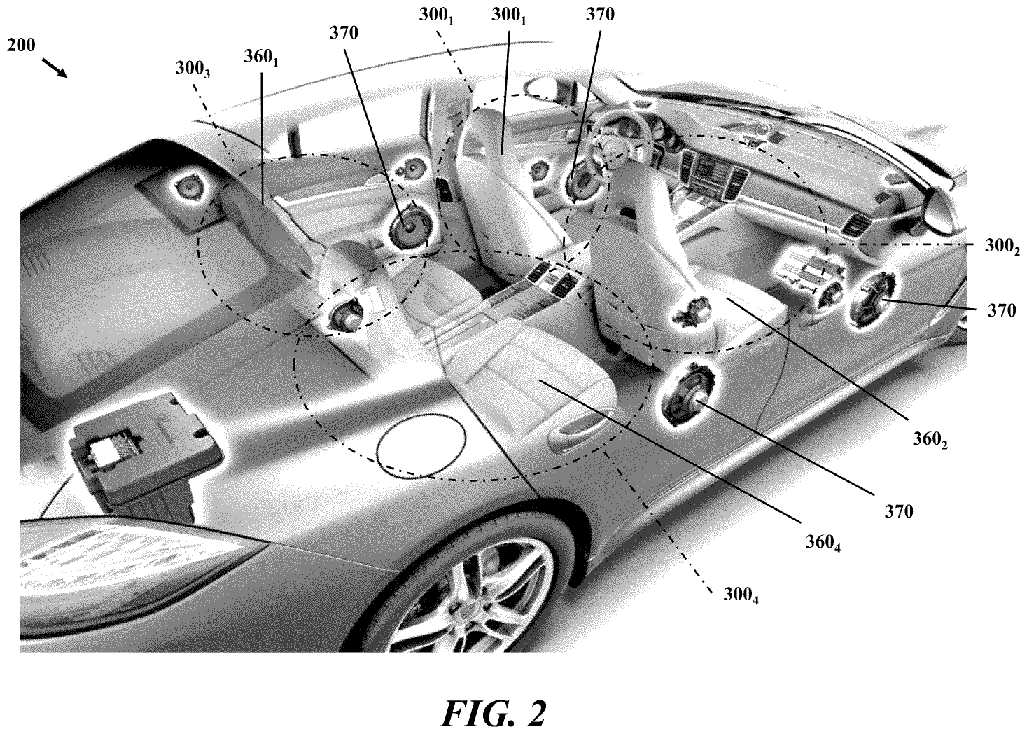

FIG. 2 is a cutaway perspective view of an exemplary embodiment of a vehicle 100 with a personalized seating zone system 200 including a plurality of personalized seating zones 300₁₋₄. Each member of the plurality of personalized seating zones 300₁₋₄ has a seat 360₁₋₄ and at least one seat system 370 associated with it. Each personalized seating zone 300 may be configurable for a specific occupant.

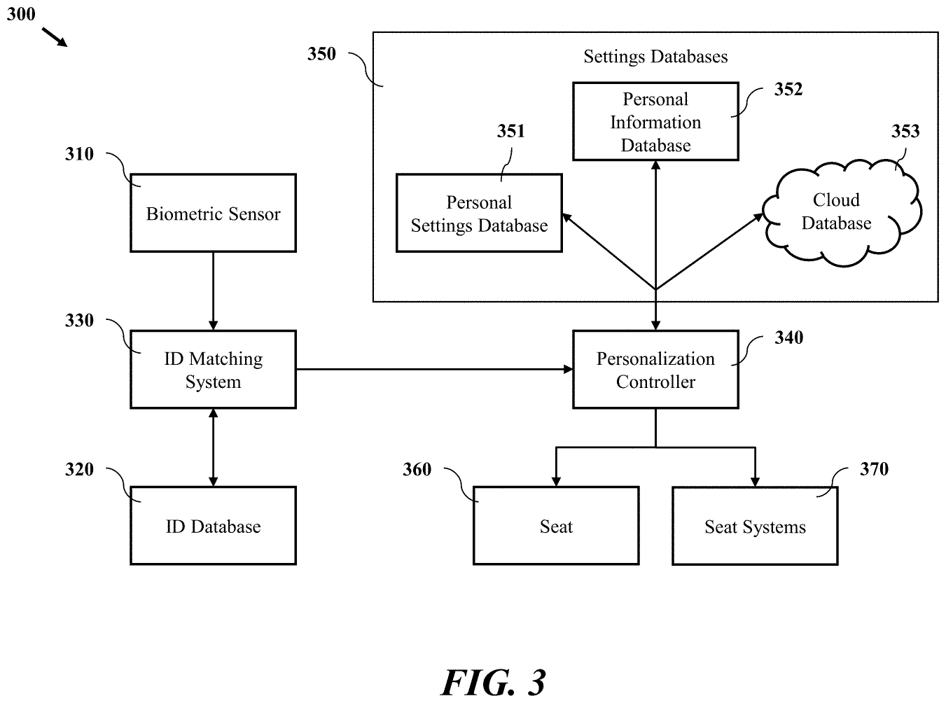

FIG. 3 is a graphical representation of an exemplary embodiment of a personalized seating zone 300. According to one embodiment, a personalized seating zone 300 comprises a biometric sensor 310, an identification database 320, an identification matching system 330, a personalization controller 340, at least one settings database 350, a seat 360, and a plurality of seat systems 370. The at least one settings database 350 may include, for example, a personal settings database 351, a personal information database 352, and a cloud database 353. The plurality of seat systems 370 may include, for example, a phone system, an audio system, a video system, a navigation system, an infotainment system, a climate system, a seating system, a cloud application system, and a browsing system including history and favorites.

According to one embodiment, the identification matching system 330 is configured to identify an occupant within the personalized seating zone 300. The identification matching system 330 may accomplish this by using the biometric sensor 310 to collect biometric data relating to the occupant and comparing the collected biometric data to stored biometric data from the identification database 320. The biometric sensor 310 may be a facial recognition camera configured to collect facial recognition data as the biometric data. The biometric sensor 310 may further be an eye tracking camera configured to collect iris recognition data and track the position of the occupant's eyes. The stored biometric data in the identification database 320 is paired with identification data and may additionally be stored iris recognition data for comparison with collected iris recognition data.

Once the occupant has been identified (i.e. the collected biometric data has been matched to a stored biometric data entry and its associated identification data entry), the personalization controller 340 may use the matched identification data to load settings for the personalized seating zone 300 based on the matched identification data. The settings to be loaded may be stored in a plurality of settings databases 350. In one embodiment, the personalization controller 340 may store and retrieve personal settings from a personal settings database 351. Personal settings may include, for example, phone system settings, navigation system settings, audio system settings, video system settings, climate control settings, browser settings, infotainment settings, and seating adjustment settings. In one embodiment, the personalization controller 340 may additionally store and retrieve personal information from a personal information database 352. Personal information may include, for example, settings history, browser history, browser favorites, and contact information. In one embodiment, the personalization controller 340 may additionally load cloud-based applications from a cloud database 353, and the personalization controller 340 may further save and retrieve settings for the cloud-based applications from the cloud database 353 or a separate database. The cloud database 353 may be independent of the vehicle 100 and may be communicated with by means of a communication system of the vehicle 100. The communication system may be, for example, a satellite radio or a cellular network.

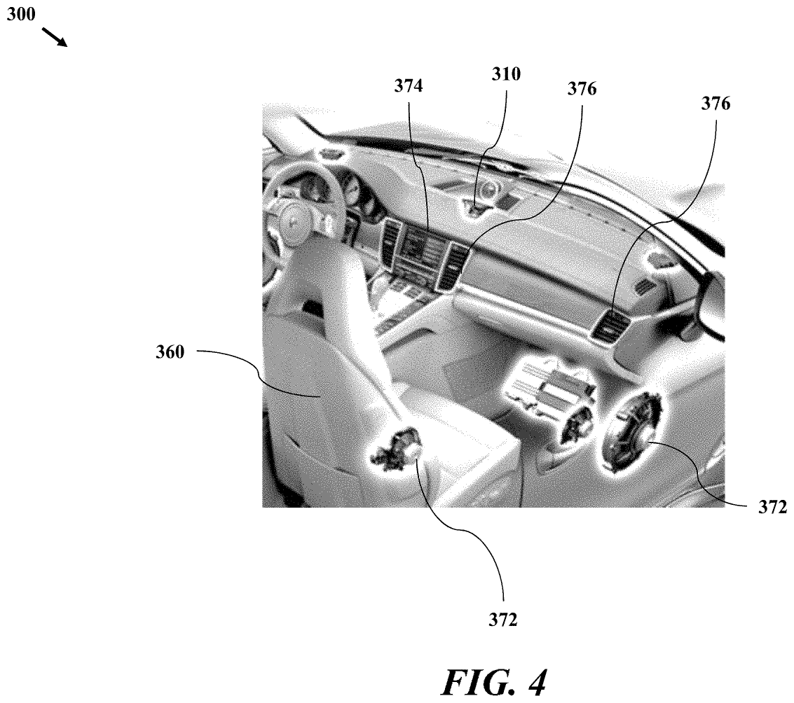

FIG. 4 is a perspective view of an exemplary embodiment of a personalized seating zone 300. The personalized seating zone 300 may include a biometric sensor 310, a seat 360, and a plurality of seat systems 370. The plurality of seat systems 370 may include, for example, a phone system, an audio system 372, a video system, a navigation system, an infotainment system 374, a climate system 376, a seating system, a cloud application system, and a browsing system including history and favorites. Many of these systems or controls for many of these systems may be integrated into a multimedia console.

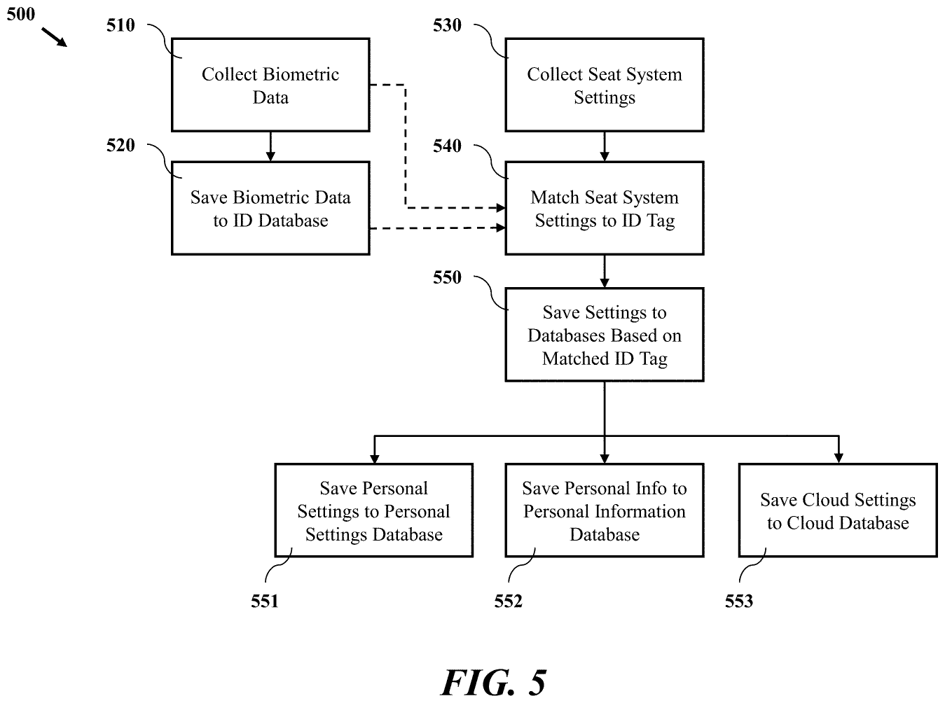

FIG. 5 is a flow chart depicting an exemplary saving process 500 for saving a user's biometric data and seat system settings to a plurality of databases for a personalized seating zone 300. The user may be an occupant of the personalized seating zone 300.

In one embodiment, at block 510, the saving process 500 collects the user's biometric data. The biometric data to be collected may be facial recognition data or iris recognition data, and the saving process 500 may collect the user's biometric data by means of a facial recognition camera or an iris recognition camera.

In one embodiment, at block 520, the saving process 500 saves the user's biometric data to an identification database. The identification database may be the identification database 320 described in FIG. 3, and the process of saving the user's biometric data to the identification database may include pairing the user's biometric data to an identification tag and saving the pairing association to the identification database.

In one embodiment, at block 530, the saving process 500 collects the user's seat system settings. The seat system settings may be configured before the saving process 500 collects them. The saving process 500 may collect the seat system settings by collecting data describing the current settings in use in the personalized seating zone 300 and/or by collecting settings configured in an application. The application may be installed, for example, on a personal computer, a portable device, or within a user interface of the vehicle 100.

The seat systems may include, for example, a phone system, an audio system, a video system, a navigation system, an infotainment system, a climate system, a seating system, a cloud application system, and a browsing system including history and favorites. The seat system settings may include personal settings, personal information, and cloud settings associated with one or more individual seat systems. Personal settings may include, for example, phone system settings, navigation system settings, audio system settings, video system settings, climate control settings, browser settings, infotainment settings, and seating adjustment settings. Personal information may include, for example, settings history, browser history, browser favorites, and contact information. Cloud settings may include, for example, settings for cloud-based applications.

In one embodiment, at block 540, the saving process 500 matches the user's seat system settings to an identification tag. Matching the user's seat system settings to an identification tag may use the identification tag from block 520, or it may directly use the user's biometric data collected at block 510 as the identification tag.

In one embodiment, at block 550, the saving process 500 saves the collected and matched seat system settings to a plurality of databases based on the matched identification tag. The saving process 500 may save the seat system settings together with the matched identification tag to facilitate loading the seat system settings at a later time. The saving process 500 may save different subsets of the seat system settings to different databases in the plurality of databases, or the saving process 500 may save multiple copies of the seat system settings as backups to different databases, including locally-stored databases and cloud-based databases. The plurality of databases may be unique to a given personalized seating zone 300 or shared between two or more personalized seating zones 300 within a personalized seating zone system 200.

According to one embodiment, the saving process 500 may save data regarding the user's seating position in the vehicle 100 and the personalized seating zone system 200. This data may be used to further categorize and customize the user's seat system settings—for example, the personalized seating zone system 200 may save a different set of seat system settings depending on which specific personalized seating zone 300 the user occupies.

At block 551, the saving process 500 may save the user's personal settings to a personal settings database. At block 552, the saving process 500 may save the user's personal information to a personal information database. At block 553, the saving process 500 may save the user's cloud settings to a cloud-based database. The personal settings database and the personal information database may be stored locally with personal seating zone 300 and the vehicle 100, while the cloud-based database may be stored on a remote server.

According to one embodiment, the saving process 500 begins at the prompting of the user. According to another embodiment, the saving process 500 begins when it recognizes a new user, i.e. a user with biometric data that does not match currently stored biometric data. The various steps of the saving process 500 may occur roughly simultaneously—that is, the saving process 500 may collect and save the biometric data and seat system settings together, such that the saving process 500 ensures that the saved data is only associated with one user.

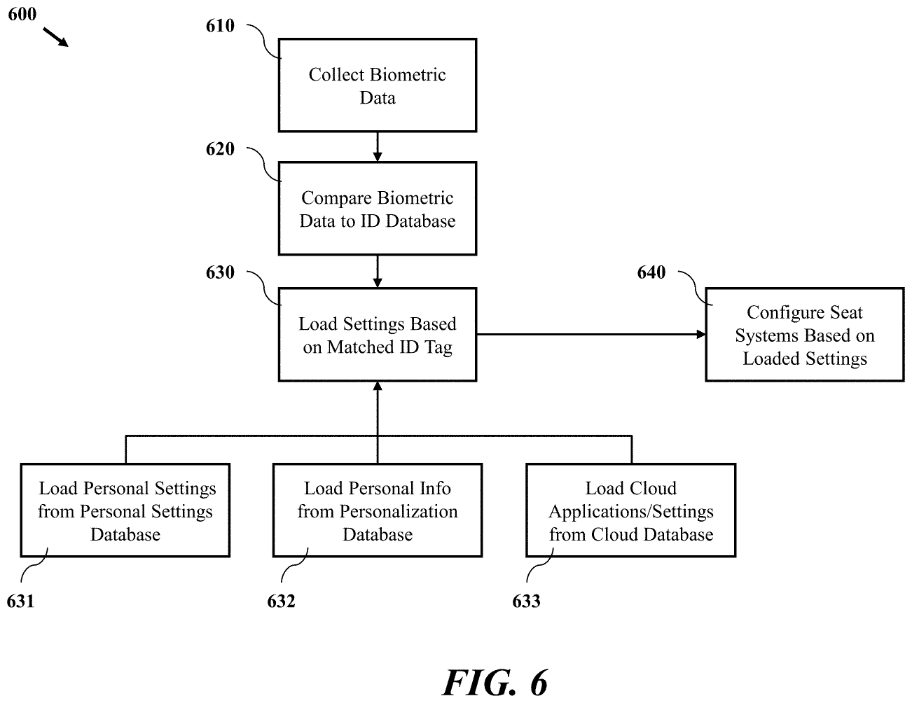

FIG. 6 is a flow chart depicting an exemplary loading process 600 for loading a user's seat system settings from a plurality of databases for a personalized seating zone 300. The user may be an occupant of the personalized seating zone 300, wherein the user has previously saved their seat system settings using the saving process 500 disclosed in FIG. 5.

In one embodiment, at block 610, the loading process 600 collects the user's biometric data. The biometric data to be collected may be facial recognition data or iris recognition data, and the loading process 600 may collect the user's biometric data by means of a facial recognition camera or an iris recognition camera. Collecting the user's biometric data may functionally use the same equipment as used at block 510 of the saving process 500.

In one embodiment, at block 620, the loading process 600 compares the collected biometric data to stored biometric data from an identification database in order to match the collected biometric data to an identification tag. The identification database may be the identification database 320 described in FIG. 3, and the process of comparing the user's biometric data to stored biometric data from the identification database may use the pairings of biometric data and identification tags that were saved as part of the saving process 500.

In one embodiment, at block 630, the loading process 600 loads seat system settings based on the matched identification tag from a plurality of databases. The seat system settings may include personal settings, personal information, and cloud settings associated with one or more individual seat systems. The seat system settings may be the user's seat system settings that were saved as part of the saving process 500. The loading process 600 may load different subsets of the seat system settings from different databases in the plurality of databases.

In one embodiment, the loading process 600 loads a different set of seat system settings depending on which specific personalized seating zone 300 within a personalized seating zone system 200 the user occupies. Alternatively, the loading process 600 may load a set of seat system settings registered to the user by a different personalized seating zone 300.

At block 631, the loading process 600 may load the user's personal settings from a personal settings database. At block 632, the loading process 600 may load the user's personal information from a personal information database. At block 633, loading process 600 may load the user's cloud settings from a cloud-based database. The personal settings database and the personal information database may be stored locally with the personal seating zone 300 and the vehicle 100, while the cloud-based database may be stored on a remote server.

The loading process 600 may either load a complete set of seat system settings or load individual system settings as needed. Similarly, the loading process 600 may be prompted by either a user entering or activating the personalized seating zone 300, or by a user using a given seat system in the personalized seating zone 300. If the loading process 600 loads seat system settings as needed, then the loading process 600 may either collect biometric data to identify the user only at the start of the session or recollect biometric data to re-verify the user for the use of each seat system.

In one embodiment, at block 640, the loading process 600 configures the seat systems associated with the personalized seating zone 300 based on the seat system settings loaded from the plurality of databases. The seat systems may include, for example, a phone system, an audio system, a video system, a navigation system, an infotainment system, a climate system, a seating system, a cloud application system, and a browsing system including history and favorites. Configuring may be, for example, changing the current settings of a seat system to match the loaded settings, populating a system database, starting media playback, or executing an application.

While this disclosure makes reference to exemplary embodiments, it will be understood by those skilled in the art that various changes in form and details may be made therein without departing from the scope of the claimed embodiments.

What is claimed is:

1. A personalized seating zone system with at least one personalized seating zone, configured to:
collect, by at least one biometric sensor, biometric data corresponding to an occupant of the personalized seating zone system;
match the collected biometric data to an identification tag to identify the occupant by comparing the collected biometric data to stored biometric data from an identification database; and
load seat system settings for a personalized seating zone within the personalized seating zone system based on the matched identification tag.

2. The personalized seating zone system of claim 1, wherein collecting biometric data includes collecting facial recognition data.

3. The personalized seating zone system of claim 1, wherein collecting biometric data includes collecting iris recognition data.

4. The personalized seating zone system of claim 3, wherein the identification database includes an iris recognition database and matching the collected biometric data includes comparing the collected iris recognition data to stored iris recognition data from an iris recognition database.

5. The personalized seating zone system of claim 1, further basing the loading of seat system settings on a seating position of the occupant.

6. The personalized seating zone system of claim 1, wherein the seat system settings include at least one from the set of phone system settings, navigation system settings, audio system settings, video system settings, climate control settings, browser settings, infotainment settings, and seating adjustment settings.

7. The personalized seating zone system of claim 1, wherein the personalized seating zone system loads personal information based on the matched identification tag.

8. The personalized seating zone system of claim 7, wherein the personal information includes at least one from the set of settings history, browser history, and browser favorites.

9. The personalized seating zone system of claim 1, wherein the personalized seating zone system loads cloud-based applications based on the matched identification tag.

10. The personalized seating zone system of claim 9, wherein the personalized seating zone system additionally loads settings for cloud-based applications.

11. A vehicle including a personalized seating zone system with at least one personalized seating zone, wherein the personalized seating zone system is configured to:
collect, by a biometric sensor, biometric data corresponding to an occupant of the personalized seating zone system;
match the collected biometric data to an identification tag to identify the occupant by comparing the collected biometric data to stored biometric data from an identification database; and
load seat system settings for a personalized seating zone within the personalized seating zone system based on the matched identification tag.

12. The vehicle of claim 11, wherein collecting biometric data includes collecting facial recognition data.

13. The vehicle of claim 11, wherein collecting biometric data includes collecting iris recognition data.

14. The vehicle of claim 13, wherein the identification database includes an iris recognition database and matching the collected biometric data includes comparing the collected iris recognition data to stored iris recognition data from an iris recognition database.

15. The vehicle of claim 11, further basing the loading of seat system settings on a seating position of the occupant.

16. The vehicle of claim 11, wherein the seat system settings include at least one from the set of phone system settings, navigation system settings, audio system settings, video system settings, climate control settings, cloud application settings, browser settings, infotainment settings, and seating adjustment settings.

17. The vehicle of claim 11, wherein the personalized seating zone system loads personal information based on the matched identification tag.

18. The vehicle of claim 17, wherein the personal information includes at least one from the set of settings history, browser history, and browser favorites.

19. The vehicle of claim 11, wherein the personalized seating zone system loads cloud-based applications based on the matched identification tag.

20. The vehicle of claim 19, wherein the personalized seating zone system additionally loads settings for cloud-based applications.
